(19) United States
(12) Patent Application Publication
(10) Pub. No.: US 2026/0160761 A1
(43) Pub. Date: Jun. 11, 2026

(54) IMMUNOSENSOR FOR ANTIGEN DETECTION VIA NON-SPECIFIC GOLD NANOPARTICLE-ANTIBODY INTERACTIONS AND SILVER ENHANCEMENT

(71) Applicant: University of Connecticut, Farmington, CT (US)

(72) Inventors: Jing Zhao, Storrs, CT (US); Yu Lei, Mansfield Center, CT (US); Chen Song, Revere, MA (US)

(21) Appl. No.: 19/410,587

(22) Filed: Dec. 5, 2025

Related U.S. Application Data
(60) Provisional application No. 63/728,371, filed on Dec. 5, 2024.

(51) Int. Cl. G01N 33/543

(52) U.S. Cl. CPC G01N 33/54346; G01N 33/54373

(57) ABSTRACT

The present disclosure relates to techniques for detection of biomolecules, and in particular to immunosensors related to gold nanoparticles.

### RELATED APPLICATIONS

The instant application claims priority to U.S. Provisional Application No. 63/728,371, filed on Dec. 5, 2024. The entire contents of the foregoing application are expressly incorporated by reference herein.

### STATEMENT REGARDING FEDERALLY SPONSORED RESEARCH

This invention was made with government support under grant number 1916213 awarded by the National Science Foundation. The government has certain rights in the invention.

### BACKGROUND OF THE INVENTION

### 1. Field of the Invention

The invention disclosed herein relates to techniques for detection of biomolecules, and in particular to immunosensors related to gold nanoparticles.

### 2. Description of the Related Art

“Immunogold,” that is, gold nanoparticles (AuNPs) conjugated with biomolecules such as antibodies and peptides, has been widely used to construct sandwiched immunosensors for biodetection. For example, immunogold was successfully used to detect Immunoglobin G (IgG), an important biomarker for SARS-CoV-2 diagnosis during the pandemic of COVID-19 as its level in serum fluctuates upon infection. However, several challenges in developing these immunoassays remain, including difficulties in finding and validating a suitable antibody, and the nonspecific interaction between the substrate and immunogold, which lowers the detection sensitivity and even causes false results.

Accordingly, there is a need in the art for improved AuNP-based immunosensors. The present disclosure meets such needs.

### SUMMARY OF THE INVENTION

In some aspects, provided herein is a kit for detecting or measuring the level of a target analyte in a biological sample, the kit including: a solid support; a capture antibody; and gold nanoparticles (AuNPs).

In some embodiments, the kit further includes a solution including silver ions. In some embodiments, the kit further includes a reducing reagent.

In some embodiments, the AuNPs are 2 nm to 15 nm, 3 nm to 10 nm, or 4 nm to 7 nm in diameter. In some embodiments, the AuNPs are about 5 nm in diameter. In some embodiments, the AuNPs are tannic acid stabilized.

In some embodiments, the solid support is a quartz crystal support. In some embodiments, the quartz crystal support is configured in a flow cell. In some embodiments, the quartz crystal support is suitable for use with a quartz crystal microbalance (QCM) instrument.

In some embodiments, the kit further includes a blocking agent. In some embodiments, the blocking agent includes bovine serum albumin (BSA). In some embodiments, the capture antibody is of the IgG class.

In some aspects, provided herein is a method of measuring the level of a target analyte in a biological sample, the method including: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample.

In some embodiments, the AuNPs are 2 nm to 15 nm, 3 nm to 10 nm, or 4 nm to 7 nm in diameter. In some embodiments, the AuNPs are about 5 nm in diameter. In some embodiments, the AuNPs are tannic acid-stabilized.

In some embodiments, the solid support is a quartz crystal support. In some embodiments, the quartz crystal support is configured in a flow cell. In some embodiments, the quartz crystal support is configured to a QCM instrument. In some embodiments, the level of AuNPs adsorbed to the solid support is measured by the QCM instrument.

In some embodiments, the method further includes one or more washing steps before and/or after steps (a), (b), (c), (d), and/or (e).

In some embodiments, after step (d) and prior to step (e), the method further includes contacting the solid support with a solution including silver ions and a reducing reagent. In some embodiments, the method further includes depositing metallic silver onto the adsorbed AuNPs. In some embodiments, the method further includes one or more washing steps after the depositing step. In some embodiments, depositing metallic silver onto the adsorbed AuNPs amplifies a signal associated with the level of the AuNPs.

In some embodiments, the target analyte is selected from the group consisting of a protein, a peptide, an antibody, an oligonucleotide, a nucleic acid, a hapten, a saccharide, a lipid, and a small molecule. In some embodiments, the target analyte is an antibody.

In some embodiments, the antibody is of the IgG class. In some embodiments, the capture antibody is of the IgG class.

In some embodiments, the solid support further includes a blocking agent immobilized thereon. In some embodiments, the blocking agent includes bovine serum albumin (BSA).

In some aspects, provided herein is a system for detecting or measuring the level of a target analyte in a biological sample, the system including: a solid support; a capture antibody; and a flow control device configured to apply one or more solutions to the solid support, the solutions including at least a first solution including gold nanoparticle (AuNPs).

In some embodiments, the solutions further include a second solution including silver ions. In some embodiments, the system further includes a reducing reagent solution.

In some embodiments, the AuNPs are 2 nm to 15 nm, 3 nm to 10 nm, or 4 nm to 7 nm in diameter. In some embodiments, the AuNPs are about 5 nm in diameter. In some embodiments, the AuNPs are tannic acid-stabilized.

In some embodiments, the solid support is a quartz crystal support. In some embodiments, the quartz crystal support is configured in a flow cell. In some embodiments, the quartz crystal support is suitable for use with a quartz crystal microbalance (QCM) instrument.

In some embodiments, the solutions further include a blocking agent solution. In some embodiments, the blocking agent includes bovine serum albumin (BSA).

In some embodiments, the capture antibody is of the IgG class.

In some aspects, provided herein is a computer program product stored on non-transitory machine-readable media, the computer program product including machine-readable instructions for controlling a process for detecting or measuring the amount of an analyte in a sample, the process including control of flow of reagents to be applied to a solid support with the reagents including at least gold nanoparticles.

In some embodiments, the process further includes control of flow of a solution including silver ions, optionally wherein the solution including silver ions further includes a reducing reagent.

In some embodiments, the analyte is an antibody, and wherein the process includes instructions for measuring the level of the antibody.

In some embodiments, the solid support is a quartz crystal microbalance (QCM) sensor and the instructions include a process for controlling operation of the QCM sensor. In some embodiments, the instructions include a process for measuring changes in the frequency of the QCM sensor based on a loading mass.

In some embodiments, the instructions include process steps including applying a sample to be analyzed, applying a reagent including gold nanoparticles, and applying a reagent including silver ions, optionally wherein the reagent including silver ions further includes a reducing reagent.

In some embodiments, the process steps further include operating a QCM sensor to measure a level of the analyte.

In some aspects, provided herein is a system for detecting or measuring the level of a target analyte in a biological sample, the system including: a solid support with a capture antibody attached thereto; and a set of reagents including a solution containing gold nanoparticles and a solution containing silver ions; wherein the capture antibody is selected to bind to a target antibody of interest.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The features and advantages of the invention are apparent from the following description taken in conjunction with the accompanying drawings in which:

FIG. 1 is a schematic diagram depicting an exemplary quartz crystal microbalance (QCM) immunosensor construction and IgG detection based on gold nanoparticles (AuNPs)-catalyzed silver enhancement. Capture antibody was first immobilized on the modified Au electrode, and the free sites were blocked with bovine serum albumin (BSA). The sample containing IgG then was introduced, and IgG molecules were captured on the substrate. In the next step, AuNPs were deposited on the free antibody molecules and catalyzed the growth of Ag particles. The frequency of the quartz crystal shows a decrease with increasing loading mass.

FIG. 2 is a graph depicting real-time resonance frequency of the QCM monitored throughout the whole sensor construction and detection process including the incubation stages of (A) polyethylenimine (PEI), (B) glutaraldehyde (GA), (C) capture antibody, (D) BSA, (E) 10 ng/mL IgG, (F) AuNPs, and (G) Ag enhancer solution. The frequency of stage (G) of silver enhancement is zoomed in and shown as the inset.

FIGS. 3A-3B are scanning electron microscopy (SEM) images of quartz crystal after the detection of 20 ng/mL IgG at (FIG. 3A) lower and (FIG. 3B) higher magnification.

FIG. 4 is a plot of Δf/Δf₀ versus the concentration of IgG sample, 0, 5, 10, 20, and 50 ng/mL. Frequency response after the Ag enhancement step was subtracted by that of the AuNPs incubation step to determine Δf caused by Ag enhancement at each IgG concentration. Ratio of Δf to that of the control sample (0 ng/mL IgG) Δf₀ was taken and plotted against the IgG concentration. A linear regression was fitted to the data at 0-20 ng/mL, and it had a slope of 0.0107 and R² value of 0.9993. Each concentration was repeated three times, and the standard deviations are represented by the error bars.

FIGS. 5A-5E are SEM images of quartz crystals after detection of IgG at (FIG. 5A) 0 ng/mL, (FIG. 5B) 5 ng/mL, (FIG. 5C) 10 ng/mL, (FIG. 5D) 20 ng/mL, and (FIG. 5E) 50 ng/mL. The scale bar of all five images is the same, as shown in (FIG. 5E).

FIGS. 6A-6C are graphs demonstrating that adsorption of AuNPs negatively correlates with the amount of IgG analyte. FIG. 6A and FIG. 6B show the number and area percentage (% area), respectively, of Ag particles produced by the detection of different concentrations of IgG (0, 5, 10, 20, and 50 ng/mL), determined from five SEM images taken at different spots for each sample. Standard deviations are represented by the error bars. FIG. 6C shows normalized Δf, number, and % area of Ag particles at different concentrations of IgG (0, 5, 10, 20, and 50 ng/mL). The Δf, number, and area (%) of Ag particles are normalized to those of the 0 ng/mL IgG sample, respectively.

FIG. 7 is a zoomed-in plot of the frequency of the QCM sensor during incubation steps of (A) PEI, (B) GA, (C) capture antibody, (D) BSA, (E) 10 ng/mL IgG, (F) AuNPs, (G) Ag enhancer solution. The small variations from (A) to (F) are resulted from the different incubation media and minor changes in the mass loading on the QCM sensor.

FIG. 8 is the UV-vis spectra of 980 μL of silver enhancer solution after being left in the dark at room temperature for 6 min (solid line) and after mixing with 20 μL of AuNPs (dash-dot line). For Ag enhancer solution only, there is no obvious peak after 6 min, indicating negligible self-growth of Ag. In contrast, its mixture with AuNPs shows a peak at around 400 nm which is ascribed to the formation of Ag shells on AuNPs. The absorption peak of AuNPs can be barely observed due to their small size, low concentration, and coverage by Ag shell. Comparing these two spectra, the catalytic effect of 5 nm AuNPs on Ag growth is demonstrated.

FIGS. 9A-9E are representative SEM images of quartz crystals after detection of IgG at (FIG. 9A) 0 ng/mL, (FIG. 9B) 5 ng/mL, (FIG. 9C) 10 ng/mL, (FIG. 9D) 20 ng/mL, and (FIG. 9E) 50 ng/mL. The scale bar for all the five images is the same, as shown in FIG. 9E. For each sample, five images were taken at randomly selected spots. The number of Ag particles and % area occupied were determined using ImageJ with a systematic grayscale and 0.001 inch² threshold applied.

FIG. 10 is the UV-vis spectra of conjugates of anti-IgG antibodies-IgG pair and AuNPs (thick solid line), anti-IgG antibodies and AuNPs (dotted line), and 5 nm AuNPs in 1×PBS (thin solid line). 50 μL of 250× concentrated anti-IgG antibodies was incubated with 250 μL of 200 ng/mL IgG at 37° C. for 1 hr, followed by incubation with 100 μL of 5 nm AuNPs at 37° C. for 1 hr. Same amount of anti-IgG antibodies alone was also incubated with 100 μL of 5 nm AuNPs at 37° C. for 1 hr. The UV-vis spectra of these two mixtures were then taken after centrifugation at 10,000 rpm for 30 min and redispersion in 1 mL 1×PBS. The peaks were marked with dashed lines and their wavelengths were labeled.

FIG. 11 is a schematic diagram of an exemplary microfluidic device including a pump and a 3D-printed flow cell, connected with tubing.

FIG. 12 is a schematic diagram of an exemplary 3D-printed flow cell. The dotted area refers to the flow pathway.

FIGS. 13A-13B are graphs demonstrating the successful detection of IgG molecules using a microfluidic device. FIG. 13A is a plot of Δf/Δf₀ versus the concentration of IgG sample, 0, 5, 10, 20, 50 ng/mL under batch mode (square) and with a 3D-printed microfluidic device (circle), and FIG. 13B is a linear fitting in the range of 0-20 ng/mL. The frequency response after Ag enhancement step was subtracted by that of AuNPs incubation step to determine Δf caused by Ag enhancement at each IgG concentration. Ratio of Δf to that of control sample (0 ng/mL IgG) Δf₀ was taken and plotted against IgG concentration. A linear regression was fitted to the data at 0 to 20 ng/mL, slope and R square value of which are shown in FIG. 13B. Each concentration was repeated three times, and the standard deviations are represented by the error bars.

FIGS. 14A-14B are graphs demonstrating the successful detection of human, mouse, and rat IgG molecules using a QCM immunosensor. FIG. 14A is a plot of Δf/Δf₀ versus the concentration of (square) mouse IgG sample, (circle) rat IgG sample, and (triangle) human IgG sample, and FIG. 14B is a linear fitting in the range of 0-20 ng/mL. Each concentration was repeated three times, and the standard deviations are represented by the error bars.

### DETAILED DESCRIPTION OF THE INVENTION

### I. Overview

Disclosed herein are methods and kits for use of gold nanoparticles (AuNP) in biomedical research applications.

Gold nanoparticles (AuNPs) have been used in biosensors for signal enhancement due to their ease of synthesis, high mass and electron density, water solubility, size-dependent optical properties, and robust conjugation with other molecules. Noteworthily, when conjugated with biomolecules (often antibodies or peptides), AuNPs show specific and strong affinity to the corresponding analytes, enabling selective targeting. Those properties of immunogold have promoted the application of immunogold in various fields, including biosensors, bioimaging, and clinical therapy. In a typical sandwiched immunoassay using immunogold, capture antibodies are immobilized on the sensor substrate for analyte binding. After the capture of analyte molecules, detection antibodies labeled with AuNPs bind with analytes at their specific binding site, forming a sandwich structure. The recognition of the analyte indicated by the binding of immunogold is then converted to a measurable signal, mostly based on surface-enhanced Raman scattering, color, or mass.

Despite the interest in the use of immunogold in biosensors, there remain several issues with the conjugation and application of immunogold including, but not limited to, loss of immuno-affinity and nonspecific binding. Finding and validation of detection antibodies with high immuno-affinity is complicated and thus requires substantial effort. Conjugation between antibodies and AuNPs can be achieved via either physisorption or chemisorption. Physisorption offers robust and facile preparation. However, it results in random orientation and soft attachment of the detection antibodies on the AuNP surface. Both of these factors attenuate the binding affinity, leading to compromised detection sensitivity. On the other hand, chemisorption brings more stable, oriented, and reproducible bioconjugates. Nevertheless, chemical modification of AuNPs with desired functional groups might affect the stability of AuNP colloids and reduce the immuno-affinity of the antibody. As for the application of immunogolds, problems with nonspecific binding exist and may bring high background or even false results. Nonspecific binding occurs between both components of immunogold (i.e., detection antibody and AuNPs) and substrate proteins including capture antibodies and blocking reagents such as Bovine Serum Albumin (BSA). When unsaturated with antibodies, the exposed Au core tends to bind with substrate proteins via various interactions, including hydrogen bonding, electrostatic, coordinative bonding, and hydrophobic interactions. On the other hand, saturated loading of detection antibodies on AuNPs requires a large excess of reagents, which not only increases the assay consumption but also reduces the binding affinity of antibodies due to the steric effect. In addition, the loosely physiosorbed detection antibody on AuNPs may potentially be replaced by other molecules, which also leads to nonspecific binding between AuNPs and substrate proteins. These problems with conjugation and application of immunogolds can be fixed to some extent by tuning the pH, blocking reagents, and coating of AuNPs, but they are impossible to eliminate completely, thus limiting the detection performance.

Accordingly, the present disclosure provides improved methods and kits for the detection of target analytes in a sample. In various embodiments, AuNPs can replace a detection antibody in a sandwiched immunoassay. Herein, AuNPs were applied solely without conjugation with detection antibodies. Without the biomolecule corona, the exposed AuNPs deposited on the capture antibodies through nonspecific interactions. In various embodiments, a decrease of AuNPs deposition on the capture antibodies was observed in the presence of target analyte. Based on this phenomenon, a sandwiched sensor for an analyte (e.g., IgG) using capture antibody as the substrate and AuNPs to replace the detection antibody is presented. Without wishing to be bound by theory, it is believed that AuNPs preferentially bind to free capture antibody compared to capture antibody-analyte complexes. Without wishing to be bound by theory, with more analyte bound to capture antibody, fewer free capture antibody molecules are available to interact with AuNPs, resulting in a reduced amount of deposited AuNPs on the solid support. Accordingly, the methods and kits of the present disclosure take advantage of the non-covalent adsorption of AuNPs, which is generally considered a limitation of immunogold in biosensors, to develop a sensitive analyte detection platform.

The present disclosure also demonstrates that the detection process can, in some embodiments, be measured using a mass-sensitive quartz crystal microbalance (QCM), since the loaded mass on the quartz crystal substrate causes a rapid shift in its resonance frequency. Additionally, it is demonstrated that the addition of a silver enhancer solution can amplify the Δf response associated with the adsorption of AuNPs, and thus improve analyte detection. In the presence of AuNPs, Ag⁺ ions were reduced to Ag⁰ and deposited onto the AuNP surface. The produced Ag particles greatly increased the mass loading on the QCM substrate, and thus increased Δf and the sensitivity.

Compared with bulk size, gold nanoparticles (AuNPs) exhibit special electronic and optical properties, such as size/shape dependent color, catalytic effect, and localized surface plasmon resonance (LSPR). It has been widely used for detection due to its ease of synthesis, high mass and electron density, water solubility, size-dependent optical properties, robust conjugation with other molecules. Noteworthily, when conjugated with biomolecules including antibodies (mostly) and peptides, the immunogold shows specific and strong affinity to the corresponding analyte targets, enabling selective detection. Those advantageous properties of AuNPs have promoted abundant studies on applying immunogold in various fields, including biosensors, bio-imaging and clinical therapy. In biosensors, the application of immunogold is usually combined with a primary antibody (also referred as capture antibody) which is immobilized on the sensor substrate for analyte target capture. After the capture of analyte molecules, the recognizing molecule like a secondary antibody on immunogold binds with analytes at a different binding site and forms a sandwich structure. The successful recognition of analyte target is followed by conversion to a measurable signal, mostly based on surface-enhanced Raman scattering, color, or mass. Great progress has been made in the conjugation and application of immunogold. For example, immunogold was successfully used to detect Immuno-globin IgG, an important biomarker of SARS-CoV-2 diagnosis during COVID-19 pandemic as its level in serum fluctuates upon the infection of COVID-19. However, there remain a few common issues with the conjugation and application of immunogold including loss of immuno-affinity, nonspecific bindings and so on. The conjugation between antibody and AuNPs can be achieved via either physisorption or chemisorption. Physisorption is the most used due to its robust preparation. However, it results in random orientation of antibody on AuNPs surface, like side-on, tail-on, head-on and flat on. In some of these orientations, the binding sites of analyte target might be blocked. Also, the soft attachment via physisorption might be replaced by other molecules in application. The binding affinity is therefore attenuated, resulting in sensitivity of biosensor being compromised. On the other hand, chemisorption based on interaction between Au core and sulfhydryl group, amine group or carbohydrate moieties brings more stable, oriented, and reproducible bioconjugates. While the chemical modification to generate required functional groups might affect the stability of AuNP colloids and reduce the immuno-affinity of antibody. The pH, concentrations of reagents, reaction time all need to be carefully optimized to get expected antibody coverage and orientation, which takes extra steps and preparation time. As for the application of immunogolds, non-specific binding always exists and brings high background noise or even false positive results. It occurs between substrate proteins with both parts of immunogold, antibody and AuNPs. Antibody is expected to bind specifically with analyte targets, however might also bind with proteins on the sensor substrate including capture antibody and blocking reagents like Bovine serum albumin (BSA). When unsaturated with antibody, the exposed Au core tend to bind with substrate proteins via various interactions, including hydrogen bonding, electrostatic, coordinative bonding, and hydrophobic interactions. The saturated loading of secondary antibody on AuNPs requires large excess of reagents which not only increases the assay consumption but also reduce the binding affinity of antibody due to steric effect. Meanwhile, as aforementioned, the loosely physiosorbed antibody on AuNPs may potentially be replaced by other molecules, which also leads to non-specific binding between AuNPs and other proteins. All these problems with conjugation and application of immunogolds can be fixed to some extent by tuning the pH, blocking reagents and coating of AuNPs, but is impossible to be eliminated completely, thus limits the detection performance.

Based on above problems using immunogold, in this work, AuNPs is applied solely, without conjugation with recognizing biomolecules. Without the biomolecule corona, the exposed AuNPs tend to deposit on capture antibody on substrate by non-specific interactions. However, in experiment as disclosed herein, a decrease of AuNPs deposition was noticed with the presence of the analyte target IgG on capture antibody. Based on this phenomenon, instead of trying to overcome the issues by non-specific interactions, advantage of it is taken for passive adsorption of AuNPs onto free capture antibody. The adsorption of AuNPs is supposed to reduce if more IgG were immobilized on capture antibody substrate. So, there is expected to be a negative correlation between loading amounts of AuNPs and IgG concentration. The detection result is interpreted as the frequency change (Δf) of quartz substrate with Quartz crystal microbalance (QCM). In recent years, QCM emerged as a robust sensing system due to its advantageous label-free mode, simple assay mechanism, ultra-high sensitivity and low manufacturing cost. In QCM instrument, a quartz crystal is embedded as the sample loading substrate. When an external voltage is applied to it, the quartz crystal oscillates owning to its piezoelectric effect. Extra loaded mass on the crystal shifts the resonant frequency according to Sauerbrey equation (1):

$$Δ {f}_{m} = - \frac{2 {f}_{0}^{2} Δ m}{\sqrt{{ρ}_{q} {μ}_{q}}} \qquad ( 1 )$$

where Δfₘ is the frequency shift due to the change in surface mass (Hz); f₀ is the fundamental resonant frequency of the crystal (Hz); Δm is the change in the mass per unit area at the crystal surface (g/cm2); ρ_{q} (g/cm3) and μ_{q} (g cm-1 s-2) are the density and shear modulus of the quartz crystal, respectively. Thus, the mass of loaded sample can be directly determined from the frequency response without any labeling of the analyte target. The frequency response changes spontaneously to the loaded mass thus enables quick detection. Its sensitivity is reported to be as low as the order of ng/cm², comparable to the gold standard technique Enzyme-linked Immunosorbent Assay (ELISA). However, the direct detection barely can be realized due to high noise background and low frequency resolution. To amplify the signal, silver enhancer solution is introduced where Ag⁺ ion is reduced to Ag⁰ by catalysis of AuNP and grows as a cluster enveloping it. The produced Ag cluster greatly enhanced the loading mass on substrate, making the frequency well resolved and enabling signal interpretation. Based on the detection mechanism, with more IgG immobilized on capture antibody substrate, fewer antibody molecules are free to interact and bind with AuNPs non-specifically, resulting in reduced amount of deposited AuNPs. As a result, less Ag clusters would produce which generates less frequency change. The correlation between the resulted frequency change caused by loading mass can be used to determine IgG concentration in sample. The mass enhancement as well as the elimination of non-specific binding by AuNPs was supposed to contribute to an ultra-sensitive and reliable detection of IgG.

Experiments were conducted to test the model. Chemicals and Materials used: Sulfuric acid, Polymethyl methacrylate (PMMA, M.W. 35000), Polyethyleneimine (PEI) were ordered from Sigma-Aldrich. Ag enhancer solution A & B were purchased from Sigma-Aldrich and mixed 1:1 for use. 30% Hydrogen peroxide, ELISA kit (contains 250× concentrated anti-mouse IgG monoclonal antibody, mouse IgG standards, coating buffer, washing buffer, blocking buffer, Assay buffer), Twenn-20 were bought from ThermoFisher Scientific. 5 nm Tannic acid stabilized gold colloid (OD 1) in 0.1 mM phosphate buffered saline (PBS) were bought from Cytodiagnostics. Glutaraldehyde (GA) was purchased from Acros Organics. All chemicals were used as received.

A 10M AT-cut gold coated quartz crystal FTQCMA-10M000-Disk (AuNPs electrode diameter 5.11 mm, crystal diameter 13.7 mm) was purchased from Fortiming Corporation.

Apparatus. Scanning electron microscopy (SEM) images were acquired with Teneo LV SEM and Verios 460L SEM. UV-vis spectra were acquired with Agilent Technologies Cary 60 UV-Vis. PMMA was sonicated to dissolve with Bransonic ultrasonic cleaner 1510R-DTH. PMMA was spin-coated on quartz crystal with a Chemat technology spin-coater KW-4A and then heated with Fisher Scientific oven 6901. Discovery-Q Sensor Innovation Development Kit including QCM instrument, incubation well plate and Data Acquisition Computer were purchased from Invitrometrix. All pipette tips and water were sterilized with Yamato SE-510 sterilizer. Quartz crystals were hydroxylated with Harrick plasma cleaner PDC-001. Incubation at 37° C. was carried out in Fisher Scientific Isotemp 637D Incubator.

Preparation of quartz crystal. QCM quartz chip was first cleaned with piranha solution (3:1 H₂SO₄:H₂O₂, v/v) to remove the surface contaminations and washed thoroughly with water. Then, 5 μl of 1% PMMA in toluene was spin coated on quartz crystal at 4000 rpm for 30 s and then heated at 60° C. for 30 min to remove solvent and improve adhesion. The prepared PMMA-coated crystal chip was stored at room temperature for immunosensor construction.

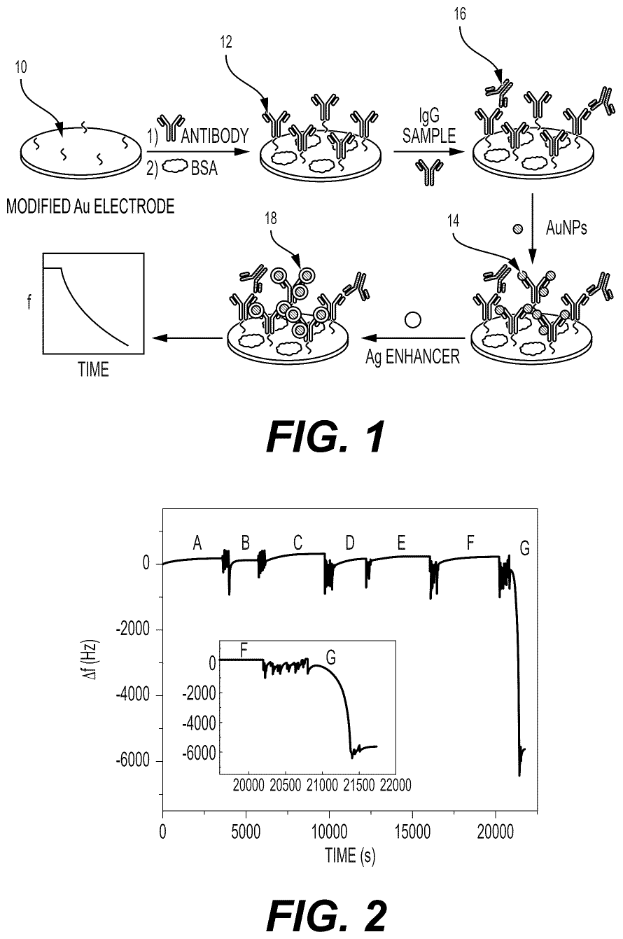

Construction of QCM sensor. As shown in FIG. 1, the prepared PMMA quartz crystal was activated with O₂ plasma for 3 min at medium power to generate hydroxyl group on its surface. The hydroxylated quartz crystal was then assembled with the incubation well plate and oscillation counter substrate, provided by the QCM kit. 200 μl of 0.6% (w/v in H2O) PEI solution was added to the well plate to incubate with the hydroxylated surface for 1 hr at room temperature, and then wash gently with water for 3 times. 200 μl of 1% (w/v in H2O) GA was then added and reacted with the amine groups of PEI at room temperature for 30 min, followed by rinsing with water three times. As a crosslinker, GA immobilized antibody in the following incubation step with 200 μl of 5× concentrated antibody in 1×PBS buffer at 37° C. for 1 hr. After washed with washing buffer (1×PBS buffer containing 0.5% (w/v) BSA and 0.1% (v/v) Tween-20) for 3 times, 250 μl of 2×PBS buffer containing 1% (w/v) BSA and 0.1% (v/v) Tween-20 is introduced into the well plate at 37° C. for 30 min to block the free sites besides capture antibody. Up to this step, the modified quartz crystal is ready as QCM sensor for IgG detection.

Referring to FIG. 1, the scheme of QCM immunosensor construction and IgG detection based on AuNPs-catalyzed silver enhancement is shown. The antibody is first immobilized on an Au electrode and the free sites were blocked with BSA. Then, the sample containing IgG was introduced, where IgG molecules were captured on a substrate. In the next step, AuNPs were deposited on free antibody molecules and catalyzed the growth of Ag clusters. The frequency of quartz crystal shows a decrease with the loading mass and helps to determine the concentration of IgG in sample.

IgG detection. A series concentrations of IgG solution were prepared, 5, 10, 20, 50 ng/ml, by diluting activated IgG stock solution with 1 PBS buffer containing 0.5% (w/v) BSA and 0.05% (v/v) Tween-20. In detection, 200 μl of IgG sample was incubated at 37° C. for 1 hr and then washed with washing buffer 3 times. Next, 200 μl of 100× diluted AuNPs solutions was added and reacted also at 37° C. for 1 hr to bind with free antibody followed by washing with washing buffer and water, both for three times. Lastly, 200 μl of silver enhancer solution was added for Ag growth. The silver staining at room temperature was stopped after 6 min by removing the Ag enhancer solution and washing twice with water. A control experiment was also carried out by substituting IgG sample with assay buffer, all other conditions remaining the same. Each experiment was repeated for at least 3 times. The frequency response was recorded during the experiment. Frequency change (Δf) in Hz of each sample were determined from the frequency responses and used to construct calibration curve.

SEM characterization of Ag cluster. After the detection of IgG samples at different concentrations, the quartz crystals were dried and saved in vacuum for SEM characterization. SEM images were taken at two magnifications 10000× and 50000× each at 5 different spots to characterize the morphology and distribution of the Ag clusters respectively. Both the count and % area of the Ag clusters was analyzed with software applying a systematic grayscale and 0.001 inch² threshold to remove the background noise.

Results. Sensor construction. The whole process of quartz crystal functionalization, sensor construction, IgG incubation and signal enhancing were monitored with QCM and plotted as frequency change with time, shown in FIG. 2.

As shown in FIG. 2, real time QCM frequency response was monitored throughout the sensor construction and detection process including incubation stage of A(a). PEI, (b). GA, (c). capture antibody, (d). BSA, (e). 10 ng/ml IgG, (f). AuNPs, and (g). Ag enhancer solution. Stage g of silver enhancement is zoomed in and shown as the insert in the graph. Frequency was subtracted to that of the beginning of the entire experiment as frequency change (Δf) with experiment running time.

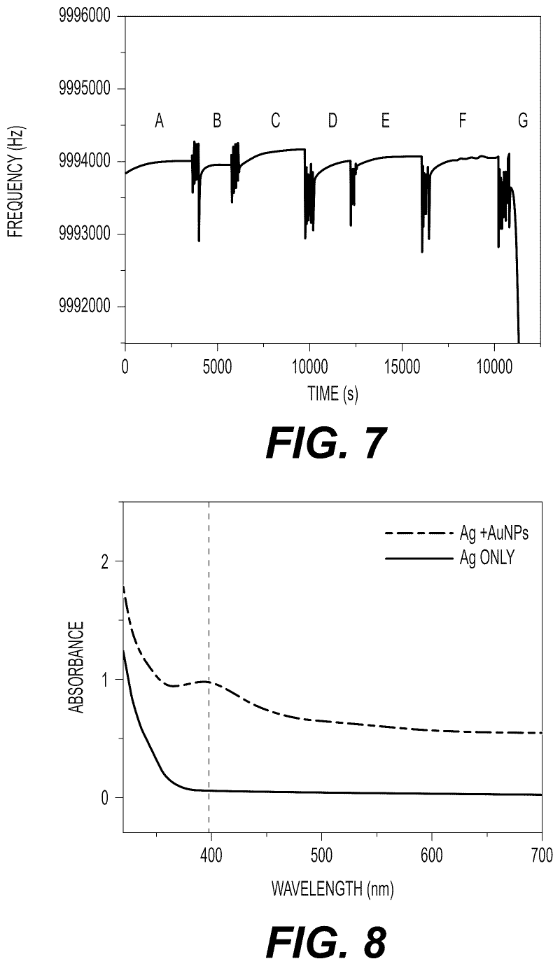

The fluctuations between each incubation steps are attributed to the pipetting and evacuating during rinsing steps. While, except stage g of Ag enhancement, each incubation step remained an almost constant frequency and only tiny variations present between each step's frequency, even for the AuNPs deposition. The low resolution of Δf after IgG capturing and AuNPs made it impossible to detect IgG concentration directly. But, at stage g, after the introduction of Ag enhancer solution into the detection system, an instant and steep frequency drop showed up as shown in the insert. The significant Δf, together with the Ag peak in UV-vis spectra of mixture of Ag enhancer solution and AuNPs in FIG. 7, proves the catalyzed growth of Ag on AuNPs. SEM images of the quartz crystal after detection of 20 ng/ml IgG at two magnifications 10000× and 75000× showed good uniformity of Ag cluster distribution. A high-resolution image of Ag clusters was taken under immersion mode with Verios 460L SEM. The Ag clusters were not in regular and identical shape, with width ranging from 150 nm to 400 nm. The variations of shape and size could be attributed to the random deposition of AuNPs and the resulted non-uniform growth of Ag on AuNPs. It might also be due to the various orientation that the cluster lies on the substrate either up straight, slantingly, or flat. For a single Ag cluster, it doesn't show a smooth contour. Instead, it seems constructs with some sub-sized pellets with a round shape. This morphology matches with the proposed mechanism that AuNPs deposits on antibody and then catalyzed the Ag growth as a shell. The significant Δf along with the SEM characterization indicates the potential application for IgG detection.

Calibration curve. Five concentrations (0, 5, 10, 20, 50 ng/ml) of IgG sample were used to construct the calibration curve, shown in FIG. 4. The ratio of Δf/Δf₀ shows a negative trend with the concentration of IgG from 0 to 50 ng/ml. At low concentrations range of 0 to 20 ng/ml, a linear regression was achieved with a slope of 0.0107 and R² value of 0.9993 referring to an excellent fitted linear relationship. The standard deviation at 0 ng/ml (0.008400) was calculated from three repeated trials and was used to calculate the limit of detection (LOD) according to the equation: LOD=3.3 σ/S, which equals 2.6 ng/ml.

As shown in FIG. 4, correlation of ratio of delta frequency response to that of control sample with the concentration of IgG sample, 0, 5, 10, 20, 50 ng/ml. Frequency response after the Ag enhancement step was subtracted to that of AuNPs incubation step as the Δf caused by Ag enhancement at each IgG concentration. Ratio of Δf to that of control sample (0 ng/ml IgG) Δf0 was taken as plotted with IgG concentration. A linear regression was simulated at low concentration range of 0 to 20 ng/ml with a slope of 0.0107 and R² value of 0.9993. Each concentration was repeated three times, and the standard deviations are shown as error bars.

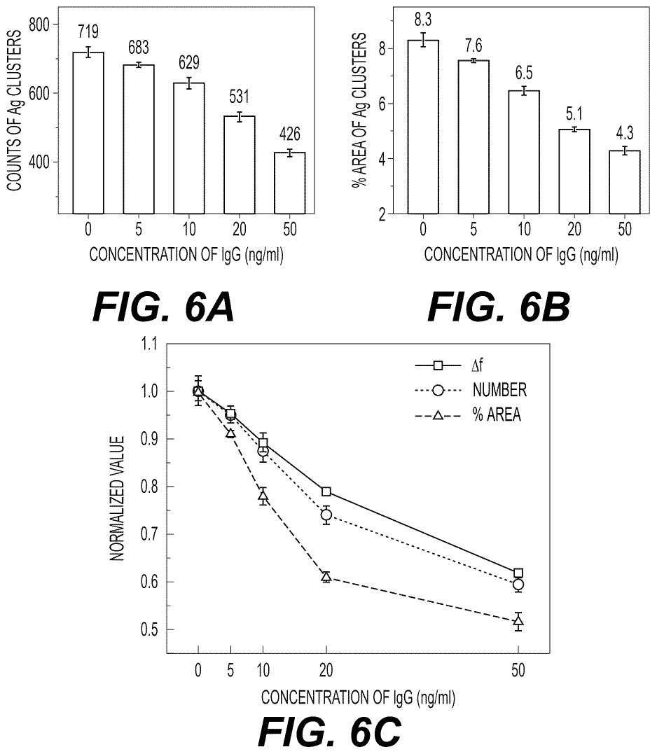

Study of Ag clusters at different IgG concentration. After detection, the quartz crystals of different IgG concentrations were characterized with SEM images at 10000× and 50000× magnification, each at 5 spots. 50000×SEM images of IgG sample with different IgG concentration 0, 5, 10, 20, 50 ng/ml showed Ag clusters in all substrates but varied in quantity. With the concentration increases from 0 ng/ml to 50 ng/ml, the number of Ag clusters showed an evident decrease. Considering the distribution is more representative at low magnification, the accurate number of Ag cluster and percentage of its occupied area in image were determined from the SEM images at 10000× using software, as shown in FIGS. 6A-6B. The count of silver clusters decreases with IgG concentration, from 722 at Ong/ml IgG to 426 at 50 ng/ml IgG concentration. Considering the size variation of Ag clusters, it might not be appropriate to simply correlate the count with the loading mass. In FIG. 6B, the % area occupied by the Ag clusters also show a decrease with the IgG concentration.

As depicted in FIGS. 6A-6B, (FIG. 6A) Counts and (FIG. 6B) area percentage (% area) of Ag clusters produced at five concentrations of IgG (0, 5, 10, 20, 50 ng/ml), determined from SEM images at 10000× magnification using software. A systematic grayscale and 0.0001 inch² threshold were applied in the software to remove the background noise.

The consistency of negative correlation between IgG concentration with Δf/Δf₀, count of Ag cluster and % area of Ag cluster can be explained as that the binding of immuno-pair antibody and IgG hampers the afterward adsorption of AuNPs to a certain extent. The adsorption of AuNPs on antibody is reported to be caused by their non-specific interactions, physical and/or chemical. The physical interaction includes electrostatic interaction between negatively charged AuNPs ligands tannic acid and positively charged region of antibody, hydrophobic attraction between the planar aromatic moieties of tannic acid and the antibody chain, and hydrogen bond. Electrostatic interaction is reported to be the initial driving force of their attachment. The charge distribution of an anti-mouse IgG monoclonal antibody has been calculated by molecular simulation, with the binding site of antibody being positively charged region. While the PDB of the antibody used in this work is told to be confidential to Sigma, so remains unknown. It may be assumed that they share similar structure and charge distribution, considering both being anti-mouse IgG monoclonal antibody. The charge distribution supports the electrostatic interaction between the antibody and tannic acid ligands of AuNPs which leads to soft attachment of AuNPs to antibody. After that, strong chemical bonds may form between Au core and thiol group of cysteine residue on antibody if any, hardening the antibody. That explains why the attached AuNPs won't be washed away during washing steps. The antibody chain usually undergoes a conformation change to make hidden cysteine residue exposed to and bind with AuNPs. However, with the presence of IgG molecules, antibody tends to bind with it thus the binding sites (positive region) of antibody are blocked from AuNPs to a certain extent. The attached IgG might also protect antibody from deformation to expose cysteine residue therefore confines the deposition of AuNPs on Fc region of antibody either. On the other hand, the conformation of neighbor antibody might be confined due to steric effect. Thus, both physical and chemical interaction are weakened with the presence of IgG molecules. This is why the AuNPs-catalyzed Ag cluster shows a negative correlation with the IgG concentration, in frequency response, count and % area of the Ag clusters.

Thus, a new immunosensor using QCM is provided. The immunosensor overcomes the long-existed non-specific binding of AuNPs in biosensors which leads to high background noise. The binding of antibody and IgG was found to reduce the adsorption of AuNPs on antibody, resulting in less catalyzed growth of Ag cluster. A decreased frequency change with IgG concentration was determined with QCM by calibration curve. This showed a dynamic detection range of 0-50 ng/ml and a good linear ship from 0 ng/ml to 20 ng/ml. The detection limit was determined to be 2.6 ng/ml. The mechanism was demonstrated by the negative correlation of IgG concentration with both count and % area of produced Ag clusters as observed in SEM images. This new method of solving common non-specific binding issue will contribute to lower the detection limit and improve the re-producibility, not only for QCM but also other immunoassays.

### II. Kits and Systems for Analyte Measurement

In some aspects, provided herein are kits for detecting and/or measuring the level of a target analyte in a biological sample. In some embodiments, the kit includes: (a) a solid support; (b) a capture antibody; and (c) gold nanoparticles (AuNPs).

In some aspects, provided herein are systems for detecting and/or measuring the level of a target analyte in a biological sample. In some embodiments, the systems include: (a) a solid support; (b) a capture antibody; and a flow control device configured to apply one or more solutions to the solid support. The solutions can include, for example, at least a first solution including gold nanoparticle (AuNPs).

The kits and systems are referred to with reference to the elements of FIGS. 1 and 12. As shown in FIG. 1, which include a solid support 10, capture antibody 12, and AuNPs 14. The flow control device 30 of a system 20, can include a flow cell, described further below and illustrated in FIGS. 11 and 12. Various other aspects of the kit, which may be understood without illustration, can include an analyte 16, and an enhancer 18. The control device may be incorporated with or include a computing system 36 that may house a computer readable medium with instructions for performing a process to detect a level of an analyte as described herein.

The term “solid support” used herein generally refers to the physical substrate or material that provides a surface for immobilizing the capture antibody in an immunoassay, e.g., an AuNP-based immunoassay presented herein. In some embodiments, the solid support is a quartz crystal support (e.g., a quartz crystal microbalance (QCM) sensor known in the art). In some embodiments, the quartz crystal support is a circular disc, wafer, or chip. In some embodiments, the quartz crystal support includes a coating for immobilization of the capture antibody and/or a blocking agent. Suitable coatings known in the art include, but are not limited to, gold, silicon dioxide, stainless steel, polystyrene, aluminum oxide, affinity-based (e.g., streptavidin-biotin or Protein A), borosilicate, cellulose, chromium, copper, gold, iron, starch, platinum, zinc oxide, silane-based (e.g., APTES (aminopropyltriethoxysilane) or GPTMS (glycidoxypropyltrimethoxysilane)), and polymers (e.g., polyethylene glycol (PEG), polystyrene, or poly(methyl methacrylate) (PMMA)). In some embodiments, the quartz crystal support is coated with PMMA.

In some embodiments, the quartz crystal support is configured in a flow cell (e.g., mounted inside a flow cell chamber or part of the flow cell body). The term “flow cell” as used herein generally refers to a chamber that allows continuous movement of a liquid sample over a quartz crystal support described herein (e.g., a QCM sensor). In some embodiments, the flow cell includes one or more inlets for introduction of one or more solutions (e.g., a solution containing a capture antibody, blocking buffer, biological sample, AuNPs, or washing buffer) into the flow cell chamber. In some embodiments, the flow cell includes one or more outlets for removal of one or more solutions (e.g., a solution containing a capture antibody, blocking buffer, biological sample, AuNPs, or washing buffer) from the flow cell chamber. In some embodiments, the flow cell is configured as a microfluidic device, wherein the microfluidic device controls the flow of one or more solutions into and out of the flow cell chamber. In some embodiments, configuration of the quartz crystal support in a flow cell allows for immobilization (e.g., adsorption or binding) of one or more reagents (e.g., capture antibody, blocking agent, AuNPs, target analyte, etc.) to the quartz crystal support. In some embodiments, configuration of the quartz crystal support in a flow cell allows real-time monitoring of immobilization (e.g., adsorption or binding) of one or more reagents (e.g., capture antibody, AuNPs, blocking agent, target analyte, etc.) to the quartz crystal support.

In some embodiments, the quartz crystal support is suitable for use with a quartz crystal microbalance (QCM) instrument. The terms “quartz crystal microbalance instrument” or “QCM instrument” as used herein to generally refer to an instrument that allows a user to monitor small mass changes on the surface of a quartz crystal support described herein (e.g., a QCM sensor). Any quartz crystal microbalance instruments known in the art can be used in the present kits and methods including, but not limited, to QCM devices available from Maxtek Inc. of Santa Fe Springs, Calif.

The terms “gold nanoparticle” and “AuNP” are used interchangeably herein and generally refer to a particle composed of elemental gold having an average diameter of 1 nm to 300 nm, 250 nm to 500 nm, 300 nm to 600 nm, 400 nm to 800 nm, 600 nm to 900 nm, or 700 nm to 1000 nm.

In some embodiments, the AuNPs are 1 nm to 100 nm in diameter. In some embodiments, the AuNPs are 1 nm to 20 nm, 10 nm to 30 nm, 20 nm to 40 nm, 30 nm to 50 nm, 40 nm to 60 nm, 50 nm to 70 nm, 60 nm to 80 nm, 70 nm to 90 nm, or 80 nm to 100 nm in diameter. In some embodiments, the AuNPs are 2 nm to 15 nm, 3 nm to 10 nm, or 4 nm to 7 nm in diameter. In some embodiments, the AuNPs are about 2 nm, about 3 nm, about 4 nm, about 5 nm, about 6 nm, about 7 nm, about 8 nm, about 9 nm, or about 10 nm in diameter. In some embodiments, the AuNPs are 2 nm, 3 nm, 4 nm, 5 nm, 6 nm, 7 nm, 8 nm, 9 nm, or 10 nm in diameter. In some embodiments, the AuNPs are about 5 nm in diameter. In some embodiments, the AuNPs are 5 nm in diameter.

AuNPs may exhibit various morphologies, including but not limited to spherical, rod-shaped, star-shaped, and dendritic structures. In some embodiments, the AuNPs are spherical.

In some embodiments, the AuNPs are stabilized by coating or functionalizing with one or more molecules that prevent aggregation. Common stabilizers include, but are not limited to, citrate ions, polymers (e.g., polyethylene glycol (PEG)), surfactants (e.g., Tween 20 or Triton X-100), alkyl thiols (e.g., dodecanethiol), and polyphenols (e.g., tannic acid). In some embodiments, the AuNPs are tannic acid-stabilized. In some embodiments, the AuNPs do not include reactive functional groups (e.g., N-Hydroxysuccinimide (NHS), maleimide, carboxyl, amine, azide, alkyne, alkyne, dibenzocyclooctyne (DBCO), and streptavidin). In some embodiments, the AuNPs are formulated as a pre-formulated suspension, concentrate, or dried nanoparticles.

In some embodiments, the kit includes one or more (e.g., 1, 2, 3, 4, or more) capture antibodies. The terms “capture antibody,” “coating antibody,” or “primary antibody” are used interchangeably herein and generally refer to an antibody that specifically binds to a target analyte in an immunoassay, e.g., an AuNP-based immunoassay presented herein. In some embodiments, the capture antibody is a polyclonal or monoclonal antibody. In some embodiments, the capture antibody is of the IgG class, of the IgM class, or of the IgA class. In some embodiments, the capture antibody is of the IgG class. In some embodiments, the capture antibody has an isotype of IgG1, IgG2, IgG3, or IgG4. In some embodiments, the capture antibody is of any species (e.g., human, mouse, or rat). In some embodiments, the capture antibody is an antibody fragment, including but not limited to, a Fab, Fab′, Fab′-SH, F(ab′)2, Fv, or scFv fragment. In some embodiments, the capture antibody specifically binds to a target analyte selected from the group consisting of a protein, a peptide, an antibody, an oligonucleotide, a nucleic acid, a hapten, a saccharide, a lipid, and a small molecule. In some embodiments, the antibody is formulated as a pre-formulated solution, concentrate, lyophilized powder, or pre-coated solid support. In some embodiments, the kit does not include a capture antibody (e.g., a user separately selects a capture antibody for use with the kit).

In some embodiments, the kit includes one or more (e.g., 1, 2, 3, 4, or more) blocking agents. The term “blocking agent” used herein generally refers to a substance that reduces nonspecific binding in an immunoassay, e.g., an AuNP-based immunoassay presented herein. In some embodiments, the blocking agent reduces nonspecific binding to the solid support (e.g., components of the biological sample that are not the target analyte). Any suitable blocking agent known in the art can be included in a kit presented herein including, but not limited to, serum, bovine serum albumin (BSA), newborn calf serum (NBCS), casein, milk (e.g., non-fat milk), and polymers (e.g., polyethylene glycol (PEG)). In some embodiments, the blocking agent includes bovine serum albumin (BSA). In some embodiments, the blocking agent is formulated as a pre-formulated solution, concentrate, lyophilized powder, or pre-coated solid support.

In some embodiments, the kit includes a solution of silver ions. In some embodiments, the kit further includes a reducing reagent. In some embodiments, the reducing reagent and the solution of silver ions in a kit presented herein are for use as a silver enhancer (e.g., to amplify a signal from AuNPs by depositing metallic silver onto the AuNP surface). In some embodiments, the solution including silver ions is a silver nitrate (AgNO₃) solution (e.g., AgNO₃ in a buffered medium). In some embodiments, the solution including silver ions is formulated as a pre-formulated solution or concentrate. In some embodiments, the kit includes silver acetate, wherein the silver acetate can be dissolved into distilled water to create a solution containing silver ions. In some embodiments, the silver acetate is formulated as a pre-formulated solution, concentrate, or crystalline solid. In some embodiments, the reducing agent is any suitable reducing reagent known in the art (e.g., hydroquinone, formaldehyde, or ascorbic acid). In some embodiments, the reducing agent is formulated as a pre-formulated solution, concentrate, or solid (e.g., crystalline solid or powder). In some embodiments, the components containing the silver reagent and the reducing reagent are kept separate in a kit provided herein. In some embodiments, the silver reagent and the reducing reagent can be combined (e.g., to create a silver enhancer solution) prior to or during use of a method provided herein, e.g., according to instructions provided with the kit. In some embodiments, the kit further includes sodium thiosulfate. In some embodiments, the sodium thiosulfate can be used in a method provided herein to stop a silver enhancement reaction and stabilize the silver deposits on the AuNPs. In some embodiments, the sodium thiosulfate is formulated as a pre-formulated solution, concentrate, or solid.

In some aspects, provided herein are kits for detecting and/or measuring the level of analyte in a biological sample. In some embodiments, the kit includes: (a) a solid support; (b) a capture antibody; and (c) gold nanoparticles (AuNPs). In some embodiments, the solid support is a quartz crystal support (e.g., a quartz crystal microbalance (QCM) sensor). In some embodiments, the quartz crystal support is coated with PMMA. In some embodiments, the AuNPs are spherical. In some embodiments, the AuNPs are about 5 nm in diameter. In some embodiments, the AuNPs are 5 nm in diameter. In some embodiments, the AuNPs are tannic acid-stabilized. In some embodiments, the capture antibody is of the IgG class. In some embodiments, the capture antibody is an anti-IgG antibody (e.g., the capture antibody specifically binds to IgG). In some embodiments, the kit further includes a blocking agent. In some embodiments, the blocking agent is BSA.

In some embodiments, the kits can include instructions for performing any of the methods described herein. The kit may be packaged in a number of different configurations such as one or more containers in a single box. The different components can be combined, e.g., according to instructions provided with the kit. The components can be combined according to a method described herein, e.g., detecting or measuring the level of a target analyte in a biological sample. In some embodiments, the kit can include one or more containers with appropriate positive and negative controls or control samples, to be used as standard(s) for detection, calibration, or normalization. The kit can further include a second container including a buffer, such as (sterile) phosphate-buffered saline, Ringer's solution, or dextrose solution. It can further include other materials desirable from a commercial and user standpoint, including other buffers, diluents, filters, and package inserts with instructions for use.

### III. Methods

In some aspects, provided herein is a method of measuring the level of a target analyte (e.g., a protein or peptide) in a biological sample (e.g., blood, plasma, urine, or saliva). In some embodiments, the method includes contacting the biological sample with a solid support including a capture antibody immobilized thereon. In some embodiments, the method includes capturing the target analyte with the capture antibody. In some embodiments, the method includes contacting the solid support with a solution including AuNPs. In some embodiments, the method includes adsorbing the AuNPs to the solid support. In some embodiments, the method includes measuring the level of the AuNPs adsorbed to the solid support. In some embodiments, the level of AuNPs is negatively correlated to the level of the target analyte in the biological sample.

In some embodiments, the method includes, in order: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. It is believed that AuNPs preferentially and unexpectedly bind to free capture antibody compared to capture antibody-analyte complexes. Without wishing to be bound by theory, with more target analyte bound to capture antibody, fewer capture antibody molecules are available to interact with AuNPs, resulting in a reduced amount of deposited AuNPs on a solid support.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the solid support is a quartz crystal support (e.g., a quartz crystal microbalance (QCM) sensor). In some embodiments, the quartz crystal support includes a coating for immobilization of the capture antibody and/or a blocking agent. In some embodiments, the coating is selected from the group consisting of gold, silicon dioxide, stainless steel, polystyrene, aluminum oxide, affinity-based (e.g., streptavidin-biotin or Protein A), borosilicate, cellulose, chromium, copper, gold, iron, starch, platinum, zinc oxide, silane-based (e.g., APTES (aminopropyltriethoxysilane) or GPTMS (glycidoxypropyltrimethoxysilane)), and polymers (e.g., polyethylene glycol (PEG), polystyrene, and poly(methyl methacrylate) (PMMA)). In some embodiments, the quartz crystal support is coated with PMMA.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the solid support is a quartz crystal support (e.g., a quartz crystal microbalance (QCM) sensor) and the quartz crystal support is configured in a flow cell (e.g., mounted inside a flow cell or part of the flow cell body). In some embodiments, the contacting of the biological sample with a solid support (step a) includes the passage of a biological sample (e.g., a sample including blood, plasma, urine, or saliva) through a flow cell (e.g., entering the flow cell chamber through an inlet of the flow cell, passage over the quartz crystal support, and exiting the flow cell chamber through an outlet of the flow cell). In some embodiments, the contacting of the solid support with a solution including AuNPs (step c) includes the passage of a solution through a flow cell. In some embodiments, the flow cell is configured as a microfluidic device, wherein the microfluidic device controls the flow of one or more solutions into and out of the flow cell chamber. In some embodiments, the quartz crystal support configured in a flow cell (e.g., mounted inside a flow cell or part of the flow cell body) is also configured to a QCM instrument (e.g., to measure the change in mass on the quartz crystal support).

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the solid support is a quartz crystal support (e.g., a quartz crystal microbalance (QCM) sensor) and the quartz crystal support is configured to a QCM instrument (e.g., to measure the change in mass on the quartz crystal support). In some embodiments, the level of AuNPs adsorbed to the solid support is measured by the QCM instrument (e.g., an increase in mass on the quartz crystal support).

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the AuNPs are 2 nm to 15 nm, 3 nm to 10 nm, or 4 nm to 7 nm in diameter. In some embodiments, the AuNPs are about 2 nm, about 3 nm, about 4 nm, about 5 nm, about 6 nm, about 7 nm, about 8 nm, about 9 nm, or about 10 nm in diameter. In some embodiments, the AuNPs are 2 nm, 3 nm, 4 nm, 5 nm, 6 nm, 7 nm, 8 nm, 9 nm, or 10 nm in diameter. In some embodiments, the AuNPs are about 5 nm in diameter. In some embodiments, the AuNPs are 5 nm in diameter. In some embodiments, the AuNPs are spherical. In some embodiments, the AuNPs include a stabilizing coating. In some embodiments, the coating is selected from the group consisting of citrate ions, polymers (e.g., polyethylene glycol (PEG)), surfactants (e.g., Tween 20 or Triton X-100), alkyl thiols (e.g., dodecanethiol), and polyphenols (e.g., tannic acid). In some embodiments, the AuNPs are tannic acid-stabilized. In some embodiments, contacting the solid support with a solution including AuNPs is performed through a flow cell configured to a microfluidic device.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the method further includes one or more (1, 2, 3, 4, or more) washing steps before and/or after steps (e). In some embodiments, the method further includes one or more (1, 2, 3, 4, or more) washing steps before and/or after steps (b). In some embodiments, the method further includes one or more (1, 2, 3, 4, or more) washing steps before and/or after steps (c). In some embodiments, the method further includes one or more (1, 2, 3, 4, or more) washing steps before and/or after steps (d). In some embodiments, the method further includes one or more (1, 2, 3, 4, or more) washing steps before and/or after steps (e). In some embodiments, the method further includes one or more (1, 2, 3, 4, or more) washing steps before step (a) (e.g., to remove contaminants from the surface of the solid support). In some embodiments, the method further includes one or more (1, 2, 3, 4, or more) washing steps after step (b) (e.g., to remove unbound biological sample from the solid support). In some embodiments, the method further includes one or more (1, 2, 3, 4, or more) washing steps after step (d) (e.g., to remove unbound AuNPs from the solid support). In some embodiments, the one or more washes are performed through a flow cell configured to a microfluidic device.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, after step (d) and prior to step (e), the method further includes contacting the solid support with a solution including silver ions and a reducing reagent (e.g., a silver enhancer solution). Suitable silver enhancer kits are known in the art and include, but are not limited to Silver Enhancer Kit (Sigma-Aldrich), Silver Enhancer Kit (High Quality) (Abcam), and LI Silver Enhancement Kit (Thermo Fisher Scientific). In some embodiments, the solution includes silver nitrate (AgNO₃). In some embodiments, the solution includes silver acetate. In some embodiments, the reducing agent is any suitable reducing reagent known in the art (e.g., hydroquinone, formaldehyde, or ascorbic acid). In some embodiments, the method further includes depositing metallic silver onto the adsorbed AuNPs after contacting the solid support with a solution including silver ions and a reducing reagent (e.g., prior to step (e)). In some embodiments, depositing metallic silver onto the adsorbed AuNPs amplifies a signal associated with the level of the AuNPs (e.g., an increase in mass on the quartz crystal support measured by a QCM instrument). In some embodiments, contacting the solid support with a solution including silver ions and a reducing reagent (e.g., a silver enhancer solution) is performed through a flow cell configured to a microfluidic device. In some embodiments, the method further includes contacting the solid support with a solution containing sodium thiosulfate, wherein the sodium thiosulfate is capable of stopping a silver enhancement reaction and stabilizing the silver deposits on the AuNPs. In some embodiments, contacting the solid support with a solution containing sodium thiosulfate is performed through a flow cell configured to a microfluidic device. In some embodiments, the method further includes one or more (1, 2, 3, 4, or more) washing steps after depositing metallic silver onto the adsorbed AuNPs (e.g., to remove unbound reducing agent and silver ions from the solid support). In some embodiments, the method further includes one or more (1, 2, 3, 4, or more) washing steps after contacting the solid support with a solution containing sodium thiosulfate (e.g., to remove unbound reducing agent, silver ions, and/or sodium thiosulfate from the solid support). In some embodiments, any of the washes are performed through a flow cell configured to a microfluidic device.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. Biological samples that can be used in the claimed method include, but are not limited to, a blood-derived sample (e.g., serum, plasma, whole blood), urine, saliva cerebrospinal fluid (CSF), cell culture supernatants, and tissue homogenates. One of ordinary skill in the art would recognize different techniques known in the art to prepare a biological sample for use in the methods provided herein, e.g., dilution, clarification, pH adjustment, and the addition of blocking agents. In some embodiments, the target analyte is selected from the group consisting of a protein, a peptide, an antibody, an oligonucleotide, a nucleic acid, a hapten, a saccharide, a lipid, and a small molecule. In some embodiments, the target analyte is an antibody. In some embodiments, the antibody is of the IgG class.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the capture antibody is a polyclonal antibody or monoclonal antibody. In some embodiments, the capture antibody is of the IgG class, of the IgM class, or of the IgA class. In some embodiments, the capture antibody is of the IgG class. In some embodiments, the capture antibody has an isotype of IgG1, IgG2, IgG3, or IgG4. In some embodiments, the capture antibody is of any species (e.g., human, mouse, or rat). In some embodiments, the capture antibody is an antibody fragment, including but not limited to, a Fab, Fab′, Fab′-SH, F(ab′)2, Fv, or scFv fragment. In some embodiments, the solid support further includes a blocking agent immobilized thereon.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the method further includes, prior to step (a), contacting a solution including the capture antibody with the solid support. In some embodiments, after contacting the solution including the capture antibody with the solid support and prior to step (a), the method further includes immobilizing the capture antibody to the solid support (e.g., through passive adsorption, covalent coupling, or affinity interactions (e.g., streptavidin-biotin)). In some embodiments, the method further includes one or more (1, 2, 3, 4, or more) washing steps after immobilizing the capture antibody to the solid support and prior to step (a) (e.g., to remove unbound capture antibody from the solid support). In some embodiments, after immobilizing the capture antibody to the solid support and prior to step (a), the method further includes contacting the solid support with a solution including a blocking agent (e.g., BSA in a buffered medium). In some embodiments, after contacting the solid support with a solution including a blocking agent and prior to step (a), the method further includes immobilizing the blocking agent to the solid support (e.g., through passive adsorption, covalent coupling, or affinity interactions (e.g., streptavidin-biotin)). In some embodiments, the method further includes one or more (1, 2, 3, 4, or more) washing steps after immobilizing the blocking agent to the solid support and prior to step (a) (e.g., to remove unbound blocking agent from the solid support). In some embodiments, any of the aforementioned steps are performed through a flow cell configured to a microfluidic device.

In some embodiments, the method includes: (a) contacting the biological sample with a solid support including a capture antibody immobilized thereon; (b) capturing the target analyte with the capture antibody; (c) contacting the solid support with a solution including AuNPs; (d) adsorbing the AuNPs to the solid support; and (e) measuring the level of the AuNPs adsorbed to the solid support, wherein the level of AuNPs is negatively correlated to the level of the target analyte in the biological sample. In some embodiments, the limit of detection of a method provided herein is 1 ng/mL to 5 ng/mL target analyte. In some embodiments, the limit of detection of a method provided herein is 1.5 ng/mL to 4 ng/mL target analyte. In some embodiments, the limit of detection of a method provided herein is 2 ng/mL to 3 ng/mL target analyte. In some embodiments, the limit of detection of a method provided herein is about 2.1 ng/mL, about 2.2 ng/mL, about 2.3 ng/mL, about 2.4 ng/mL, about 2.5 ng/mL, about 2.6 ng/mL, about 2.7 ng/mL, about 2.8 ng/mL, about 2.8 ng/mL, or about 2.9 ng/mL target analyte. In some embodiments, the limit of detection of a method provided herein is 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.8 ng/mL, or 2.9 ng/mL target analyte. In some embodiments, the limit of detection of a method provided herein is about 2.6 ng/mL target analyte. In some embodiments, the limit of detection of t a method provided herein is 2.6 ng/mL target analyte.

### IV. Definitions

Definitions provided herein are offered to aid in describing particular embodiments, and are not to be construed as limiting of the claimed invention. Unless otherwise defined, all technical and scientific terms used herein are to be construed as commonly understood by one of ordinary skill in the art to which this invention belongs. If there is an apparent discrepancy between the usage of a term in the art and its definition provided herein, the meaning of the term shall be construed in a manner most favorable to the invention, such as where the definition provided within the specification shall prevail.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “about” or “approximately” means within 5%, or more preferably within 1%, of a given value or range.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

In order that the present disclosure may be better understood, the following examples are set forth. These examples are for purposes of illustration only and are not to be construed as limiting the scope of the present disclosure in any manner.

### EXAMPLES

### Example 1

This example demonstrates the successful detection of IgG molecules using a Quartz Crystal Microbalance (QCM) immunosensor.

### I. Materials and Methods

Chemicals and Materials. Sulfuric acid, poly(methyl methacrylate) (PMMA, MW of 35 000), and polyethylenimine (PEI, MW of 25000) were purchased from Sigma-Aldrich. Ag enhancer solutions A and B were purchased from Sigma-Aldrich and mixed at 1:1 for use. 30% Hydrogen peroxide, ELISA kit (contains 250× concentrated antimouse IgG monoclonal antibody, mouse IgG standards, coating buffer, washing buffer, blocking buffer, and assay buffer), and Tween-20 were acquired from ThermoFisher Scientific. The 5 nm tannic acid stabilized gold colloid (OD 1) in 0.1 mM phosphate buffered saline (PBS) was obtained from Cytodiagnostics. Glutaraldehyde (GA) was purchased from Acros Organics. The 10 MHz AT-cut gold-coated quartz crystal FTQCMA-10M000-Disk (Au electrode diameter 5.11 mm, crystal diameter 13.7 mm) was purchased from Fortiming Corp. All chemicals and materials were used as received.

Instrumentation. SEM images were acquired with a Teneo LV SEM and a Verios 460L SEM and analyzed using ImageJ 1.54f. UV-vis spectra were acquired with an Agilent Technologies Cary 60 UV-vis. A Discovery-Q Sensor Innovation Development Kit including QCM instrument, incubation well plate, and Data Acquisition Computer were purchased from Invitrometrix. All pipet tips and water were sterilized with a Yamato SE-510 sterilizer prior to use. Quartz crystals were hydroxylated with the Harrick plasma cleaner PDC-001. Incubation at 37° C. was carried out in a Fisher Scientific Isotemp 637D incubator.

Preparation of Quartz Crystal. The QCM chip was first cleaned with piranha solution (3:1 H₂SO₄:H₂O₂, v/v) to remove the surface contaminations and then washed thoroughly with water. Next, 5 μL of 1% PMMA in toluene was spin coated onto the quartz crystal at 4000 rpm for 30 s and then heated at 60° C. for 30 min to remove solvent and improve adhesion. The prepared PMMA-coated quartz crystal was stored in a Petri dish at ambient conditions before sensor construction.

Construction of QCM Sensor. As shown in FIG. 1, the PMMA-coated QCM chip was activated with O₂ plasma for 3 min at medium power to generate hydroxyl groups on its surface. The hydroxylated chip was then assembled with the incubation well plate and oscillation counter substrate, provided in the QCM kit. 200 μL of 0.6% (w/v in H₂O) PEI solution was added to the well plate to incubate with the hydroxylated chip for 1 h at room temperature. The chip was then washed gently with water three times. 200 μL of 1% (w/v in H2O) GA was then added and reacted with the amine groups of PEI at room temperature for 30 min, followed by rinsing with water three times. As a cross-linker, GA helps to immobilize the antibody in the following incubation step with 200 μL of 5× concentrated antibody in 1×PBS buffer at 37° C. for 1 h. After being washed with washing buffer (1×PBS buffer containing 0.5% (w/v) BSA and 0.1% (v/v) Tween-20) three times, 250 μL of 2×PBS buffer containing 1% (w/v) BSA and 0.1% (v/v) Tween-20 was introduced into the well plate at 37° C. for 30 min to block the free sites in addition to the capture antibody. Up to this step, the modified QCM chip is ready for IgG detection.

IgG Detection. IgG solutions of 5, 10, 20, and 50 ng/mL were prepared by diluting activated IgG stock solution with 1×PBS buffer containing 0.5% (w/v) BSA and 0.05% (v/v) Tween-20. For detection, 200 μL of IgG sample was added to the QCM chip and incubated at 37° C. for 1 h. The chip was then washed with washing buffer three times. Next, 200 μL of 100× diluted AuNPs solutions was added and reacted also at 37° C. for 1 h to bind with free antibodies, followed by washing with washing buffer and water, each three times. Last, the sensor was taken out of the incubator, and 200 μL of silver enhancer solution was added for Ag growth. The silver staining reaction at room temperature was stopped after 6 min by removing Ag enhancer solution and washing twice with water. A control experiment was also carried out by substituting IgG sample with the assay buffer, keeping all other conditions the same. Each experiment was repeated at least three times. The frequency response was monitored during the whole experiment. Frequency change (Δf) in Hz of each sample was determined and used to construct the calibration curve.

SEM Characterization of Ag Particles. After the detection of IgG samples at different concentrations, the quartz crystals were dried and preserved in a vacuum for SEM characterization. SEM images were taken at different magnifications, each at five different spots to characterize the morphology and distribution of the Ag particles. Both the number and the area percentage of the Ag particles were determined with ImageJ software applying a systematic grayscale and 0.001 in.² threshold.

### II. Results

Sensor Performance. During the whole process of quartz crystal functionalization, sensor construction, IgG incubation, AuNPs incubation, and signal enhancing by Ag growth, the frequency of the QCM chip was monitored and plotted with time, shown in FIG. 2. The IgG concentration in this FIG. 2 was 10 ng/mL. The drastic fluctuations between each incubation step are caused by pipetting and evacuating during rinsing. The frequency response at stages a-f was zoomed in and shown in FIG. 7. From FIG. 2 and FIG. 7, except stage g of Ag enhancement, the frequency of each incubation step showed only small variations, even for AuNPs deposition. However, at stage g, after the introduction of Ag enhancer solution into the incubation well, an instant and steep frequency drop was observed as shown in the inset in FIG. 2. The significant frequency change, together with the Ag peak in the UV-vis spectra of a mixture of Ag enhancer solution and AuNPs in FIG. 8, proves the catalyzed growth of Ag particles by AuNPs.

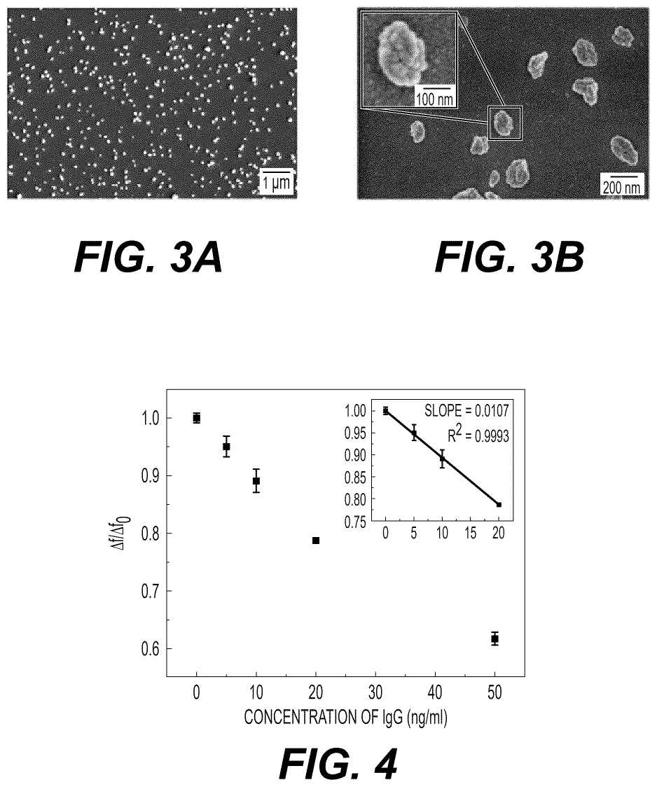

The SEM images of the quartz crystal after detection of 20 ng/mL IgG at two magnifications are shown in FIGS. 3A-3B. The SEM image at low magnification presented in FIG. 3A shows good uniformity of Ag particle distribution. From the zoomed in image in FIG. 3B, the Ag particles are not in regular and identical shapes, with sizes ranging from 150 to 400 nm. The variations of shape and size could be attributed to the random deposition of AuNPs on free capture antibodies and the resulted irregular growth of Ag around the AuNPs. It might also be due to the various orientations of the Ag particles on the quartz crystal substrate, such as straight up, slanted, or flat. The Ag particles do not have a smooth contour (see the inset in FIG. 3B). Instead, it seems that they were constructed with many spherical subdomains. This morphology can be attributed to the growth mechanism that small spherical AuNPs deposited on the antibodies and then catalyzed the Ag growth as a shell surrounding the AuNP-decorated antibodies, thus supporting the proposed sensing method.

Calibration Curve. Five concentrations (0, 5, 10, 20, and 50 ng/mL) of IgG samples were used to construct the calibration curve, shown in FIG. 4. Δf is the difference in the frequency for each IgG sample after 6 min of Ag growth compared to that before the addition of silver enhancer solution. Δf₀ represents Δf of the control experiment in the absence of IgG. The ratio of Δf/Δf₀ was taken to minimize the frequency response differences across different QCM chips, caused by the variations in the intrinsic resonance frequency of the chip determined by manufacturing. Δf/Δf₀ showed a clear decrease as the concentration of IgG increased from 0 to 50 ng/mL. At the concentration range of 0-20 ng/mL, a linear regression was achieved with a slope (S) of 0.0107 and R2 value of 0.9993. The standard deviation (σ) at 0 ng/mL (0.008400) was calculated from three repeated trials and was then used to calculate the limit of detection (LOD) according to the equation LOD=3.3σ/S, which equals 2.6 ng/mL.

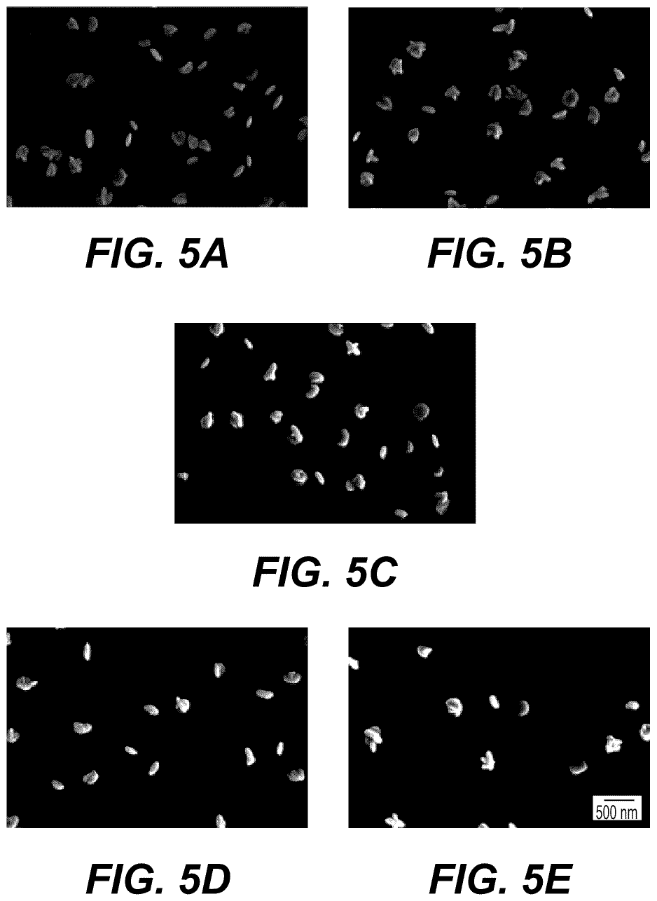

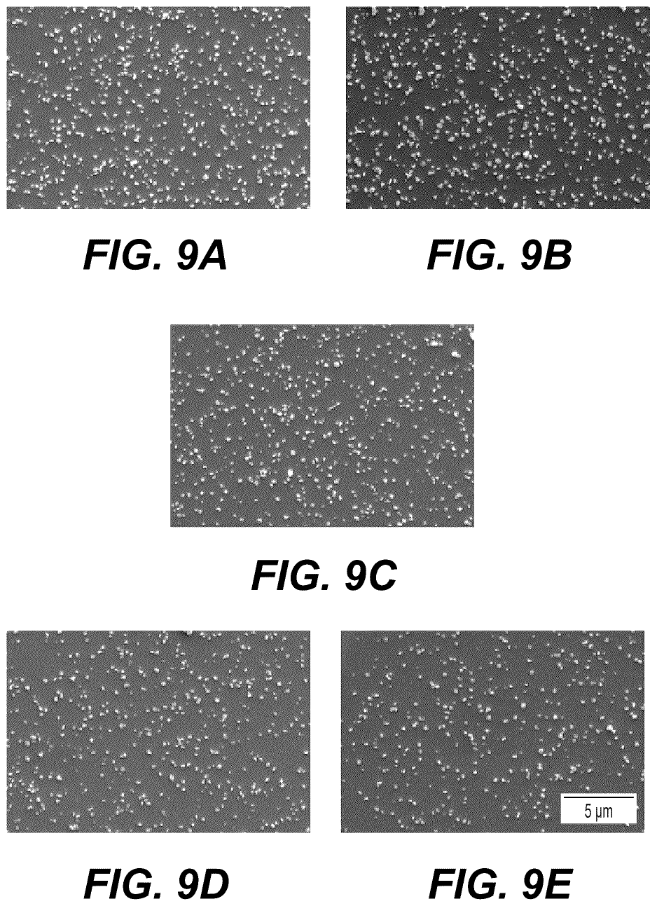

Ag Particle Growth at Varying IgG Concentrations. After detection, the quartz crystals incubated with different concentrations of IgG were characterized with SEM, each at five spots. Representative SEM images of IgG sample with different IgG concentrations of 0, 5, 10, 20, and 50 ng/mL are shown in FIGS. 5A-5E. Ag particles are observed in all of these substrates but vary in quantity. When the concentration increases from 0 to 50 ng/mL in FIGS. 5A-5E, the number of Ag particles shows an evident decrease. Considering the distribution of Ag particles is more representative when imaged in a larger area, the accurate number of Ag particles and percentage of their occupied area in the image were determined from the SEM images at lower magnification (representative images shown in FIGS. 9A-9E) using ImageJ software, averaged by the data from five randomly chosen spots in each sample. The number of Ag particles in the microscopic area (287.6 μm² according to SEM) decreases with increasing IgG concentration, from 719 at 0 ng/mL IgG to 426 at 50 ng/mL IgG concentration, as shown in FIG. 6A. The % area occupied by the Ag particles was also calculated, and it showed a similar decreasing trend with increasing IgG concentration (FIG. 6B). These trends of number of Ag particles and % occupied area are consistent with those of the frequency response as shown in FIG. 6C, which again validates the proposed detection mechanism in FIG. 1.

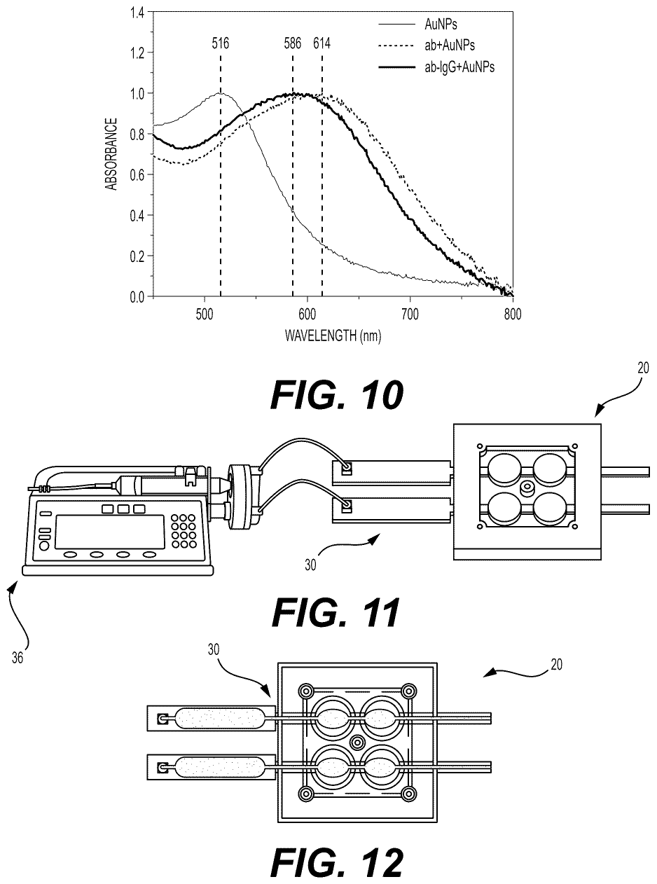

Without wishing to be bound by theory, the consistency of the negative correlation between IgG concentration and Δf/Δf₀, number of Ag particles, and % area of Ag particles may be explained by that the binding of immuno-pair (i.e., anti-IgG antibody and IgG) hampers the subsequent adsorption of AuNPs. The adsorption of AuNPs on antibody is reported to be caused by their nonspecific interactions, physical and/or chemical. The physical interactions include the electrostatic interaction between negatively charged tannic-acid-capped AuNPs and the positively charged region of the anti-IgG antibody, hydrophobic attraction between the planar aromatic moieties of tannic acid and the antibody chain, and hydrogen bond. Electrostatic interaction is reported to be the initial driving force of their attachment. The charge distribution of an anti-mouse IgG monoclonal antibody has been reported in previous work, and it was found that the binding sites of the antibody are positively charged. The structure of anti-mouse IgG monoclonal antibody used in this work remains confidential by the ELISA kit supplier, but it is assumed to share a charge distribution similar to that in the reference above, considering their similarity. Therefore, electrostatic interaction occurs between the anti-IgG antibody and tannic acids on AuNPs, which leads to soft attachment of AuNPs to the antibody. After that, strong Au—S chemical bonds may form between the Au core and the thiol group of the cysteine residue on the antibody if any, hardening the antibody-AuNPs conjugates. The antibody chain usually undergoes a conformation change to make the hidden cysteine residue exposed to and bind with AuNPs. However, in the presence of IgG molecules, IgG binds to positively charged binding sites of the antibody, blocking the further deposition of AuNPs. The attached IgG might also protect the antibody from deformation to expose cysteine residue, which therefore confines the deposition of AuNPs onto the Fc region of the antibody. On the other hand, the conformation of the neighbor antibody might also be confined due to the steric effect. Thus, both physical and chemical interactions between anti-IgG and AuNPs are weakened in the presence of IgG molecules. The weakening effect on the interaction between anti-IgG antibody and AuNPs was further supported by the UV-vis spectra of conjugates of anti-IgG antibodies and AuNPs, as shown in FIG. 10. With the preincubation of anti-IgG antibodies and IgG, their conjugates with AuNPs exhibit an absorption peak at a shorter wavelength (586 nm) compared to that of anti-IgG antibodies with AuNPs (614 nm), whereas AuNPs alone have a peak at 516 nm. The spectral change when AuNPs are mixed with anti-IgG antibodies suggests that they were attached and aggregated onto anti-IgG antibodies, causing a red shift in the extinction peak. The shorter wavelength of AuNPs mixed with IgG-anti-IgG pairs can be inferred as less aggregation of AuNPs, indicating less interaction between IgG-anti-IgG pairs and AuNPs compared to that of anti-IgG antibodies to Au NPs.

This inverse relationship between the binding of IgG and adsorption of AuNPs to anti-IgG antibody leads to a lower amount of AuNPs on the quartz crystal substrate when increasing the IgG concentration. Since Ag particle growth was catalyzed by AuNPs, the lower was the amount of AuNPs, the lower was the number of Ag particles formed. Therefore, the Ag particles' growth shows a negative correlation with the IgG concentration, in frequency response, number, and area percentage of the Ag particles.

### III. Conclusion

In this work, an immunosensor using QCM is developed to overcome the long-existing issue of nonspecific binding of immunogold in biosensors, which leads to high background and sometimes even false results. The binding of IgG to the antibody was found to reduce the subsequent adsorption of AuNPs onto the antibody, resulting in less catalyzed growth of Ag particles. A decreased frequency change with increasing IgG concentration was observed with QCM measurements. The frequency response showed a dynamic detection range of 0-50 ng/mL and a good linearity from 0 to 20 ng/mL. The detection limit was determined to be 2.6 ng/mL. The proposed binding mechanism was further supported by the negative correlation of IgG concentration with both the number and the % area of Ag particles formed as well as the UV-vis spectra of AuNPs bioconjugates. This new method of solving common nonspecific binding issues will contribute to lowering the detection limit and improving the reproducibility in immunoassays. It also provides a new sensing strategy, complementing the conventional immunoassays relying on antibody-coated AuNPs.

### Example 2

This example demonstrates the successful detection of IgG molecules using a microfluidic device.

### I. Methods

A 3D-printed microfluidic device was designed using Autodesk Fusion which has incubation wells with two of them sharing the same flow (FIG. 12). The microfluidic device (FIG. 11) was connected to a four-channel pump with a 10 mL syringe loaded on each channel as air pump and washing solution source. The two wells sharing the same flow are treated as duplicates, and an average was calculated from which as the frequency response. One of the flows was used for detection of 0 ng/mL IgG as an assay control, while the other one was used for detection of 5-50 ng/mL IgG. Each concentration was repeated three times, the standard deviation from which was treated as reproducibility. A Δf/Δf₀ value was generated from each run and used for calibration curve construction.

The experiment was conducted as follows: a) Coat QCM chips with polymethyl methacrylate (PMMA) and activate with Oxygen plasma; b) Assemble the instrument and connect it to ethernet; c) Go to http://ivmx-discovery.local/# and start running oscillator mode; d) Pump in 480 μL of 0.6% (w/v) PEI solution at 500 ul/min and incubate for 1 hr at R.T.; e) Pump in air at 500 μL/min for 1 min to blow all the leftover solution out; f) Wash with water by pumping at 1500 μL/min for 1 min; g) Pump in air at 500 μL/min for 1 min to blow all the leftover solution out; h) Pump in 1% (w/v) glutaraldehyde (GA) at 500 μL/min and incubate for 30 min at R.T.; i) Repeat step e-g; j) Pump in 50× diluted capture antibody at 2000 μL/min and incubate for 1 hr at R.T.; k) Repeat step e-g (replace water with washing buffer); 1) Pump in blocking buffer at 2000 μL/min and incubate for 30 min at R.T.; m) Repeat step e-g (replace water with washing buffer); n) Pump in IgG at 2000 μL/min and incubate for 1 hr at R.T.; o) Repeat step e-g (replace water with washing buffer); p) Pump in 10× diluted AuNPs solution at 2000 μL/min and incubate for 1 hr at R.T.; q) Repeat step e-g (replace water with washing buffer); r) Pump in Ag enhancer solution at 2000 μL/min and incubate for 6 min at R.T. in dark; s) Repeat step e-f (replace water with washing buffer); t) Repeat step e-f, u) Repeat step e-f, v) Stop acquisition.

### II. Results

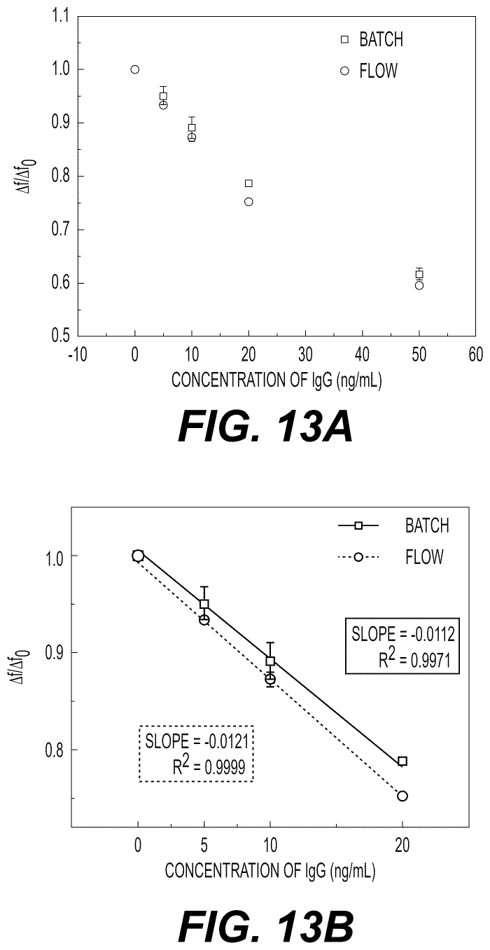

As shown in FIGS. 13A-13B, slightly lower Δf/Δf₀ values were noticed for all five concentrations under batch mode and flow mode. While a microfluidic device offers more reproducible results, indicated from the negligible error bars of flow mode, compared with those of batch mode, which can be attributed to the more controllable and efficient washing between steps. Limit of detection (LOD) of IgG using microfluidic device was also calculated using the equation LOD=3.3 σ/S, which equals 0.626 ng/mL. Compared with the LOD under batch mode 2.6 ng/mL, both the sensitivity and reproducibility were improved by using 3D-microfluidic system.

### Example 3

This example demonstrates the successful detection of IgG molecules from different species using a Quartz Crystal Microbalance (QCM) immunosensor.

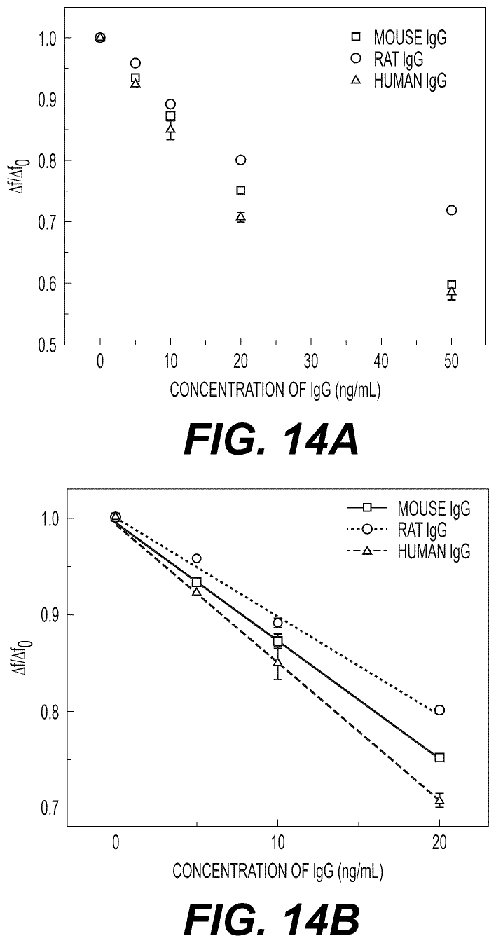

Human, mouse, and rat IgG were detected according to the methods of Example 1. The results from this experiment are provided in FIGS. 14A-14B. For each species, Δf/Δf₀ showed a clear decrease as the concentration of IgG increased from 0 to 50 ng/mL. Accordingly, the QCM immunosensor is capable of measuring the concentration of IgG across multiple species.

Having introduced aspects of the technology, some additional features and embodiments are now presented.

The technology may operate within a system that includes mechanical, electrical, optical, and computing elements. A sensor platform may include a quartz crystal support mounted within a flow cell, a microfluidic housing that defines one or more flow pathways, and a set of ports that introduce and discharge liquids. A pump, a valve array, or a pressurized reservoir may drive movement of each reagent across the quartz crystal support. A structural frame may hold the flow cell, the pump, and any tubing. A cartridge interface may receive a removable flow cell. A set of detectors may connect to the quartz crystal support to deliver a driving signal and to measure a resonance response. A control board may condition each signal and may convert each measurement into digital form.

A system may incorporate storage vessels for AuNPs, wash buffers, capture antibody solutions, biological samples, and silver-ion reagent solutions. Each storage vessel may connect through tubing to the flow cell. Heaters, thermoelectric devices, or temperature sensors may maintain the temperature of the flow cell. A timing unit may regulate the duration of each incubation, wash, or enhancement step. A filtration module may condition incoming biological samples. A waste reservoir may receive spent solutions.

The technology may further operate within a computing environment that manages the sequence of reagent delivery and the acquisition of resonance-frequency data. A computing device may include a processor, a memory, a communication interface, and non-volatile storage. The processor may execute instructions that control the pump, valve array, or microfluidic actuators. The processor may also execute instructions that sample, transform, and store QCM data. The memory may hold calibration constants, threshold values, sample identifiers, and flow-sequence data. The computing device may reside within the sensor housing or may be remote from the housing.

A computing device may include firmware, middleware, or application software that directs the hardware. Software modules may set reagent-flow rates, may adjust incubation times, may trigger washing events, and may govern introduction of AuNPs and silver-ion solutions. Software modules may process raw resonance-frequency information into values that represent mass loading. Additional modules may perform noise reduction, curve fitting, or statistical analysis. Modules may evaluate a negative correlation between deposited AuNPs and analyte concentration to generate a concentration value for the sample. A data-logging module may store time-stamped data in a relational or non-relational database. A communication module may transmit results to a local workstation or a cloud resource.

Generally, “software” may be provided as at least one computer program product stored on non-transitory machine-readable media. Generally, software will enable machine oriented execution of methods and processes as described herein or as may be contemplated by one skilled in the art.

A distributed computing architecture may support multiple sensors. A server may coordinate assay scheduling, flow-cell preparation, and calibration routines. A networked device may receive QCM data from each sensor and may apply machine-learning algorithms to refine detection thresholds or to identify drift in system performance. A software library may expose an application programming interface that allows future analytical tools to access raw or processed data. A virtual machine or a containerized environment may run signal-processing code that operates independently of other system components.

The technology may also operate with firmware that executes at the edge of the system. Firmware may manage low-level communication with oscillation drivers, frequency counters, or analog-to-digital converters. Firmware may govern safe sequencing of fluidic actuators. Firmware may monitor pump load, flow resistance, or temperature. Firmware may maintain compliance with timing constraints necessary for accurate measurement.

A system may include any combination of these mechanical, electrical, and computing components. Any component described herein may operate alone or in combination with other components to implement measurement of analytes by adsorption of AuNPs and silver enhancement on a quartz crystal support.

Generally, the technology may be used in a variety of settings. For example, a system based on adsorption of gold nanoparticles on free antibody regions may operate in a diagnostic laboratory to measure concentrations of immunoglobulins in blood, plasma, serum, saliva, tear fluid, or urine. A system based on this technology may measure biomarkers associated with infectious disease, autoimmune disease, metabolic disease, or allergic response. A system based on this technology may operate in a hospital or clinic to support point-of-care testing that provides timely evaluation of patient status. A system based on this technology may operate in a treatment facility to monitor variation of antibody levels during therapy.

A system based on adsorption of gold nanoparticles on free antibody regions may operate in a research laboratory to quantify antibody production during immunization studies, protein expression studies, or cell-culture assays. A system based on this technology may evaluate binding characteristics of antibody fragments, engineered antibodies, or synthetic affinity reagents. A system based on this technology may support detection of proteins, peptides, haptens, nucleic acids, or lipids when suitable capture molecules are immobilized on a quartz crystal support.

A system based on adsorption of gold nanoparticles on free antibody regions may operate in pharmaceutical development to assess drug-antibody interactions, drug-induced modulation of immune response, or formation of immune complexes. A system based on this technology may measure quality attributes of biologic therapeutics including batch-to-batch variation of immunoglobulin concentration in production streams. A system based on this technology may operate in a manufacturing environment to evaluate purity of process intermediates or to monitor removal of antigen contaminants.

A system based on adsorption of gold nanoparticles on free antibody regions may operate in a microfluidic cartridge configured for field deployment. A system based on this technology may support environmental monitoring of antigenic contaminants in water, soil, or air when appropriate capture molecules are used. A system based on this technology may operate in veterinary diagnostics to measure immunoglobulin levels in animal samples, including samples from livestock, companion animals, or laboratory animals. A system based on this technology may support food-safety testing to detect antigenic markers associated with pathogens in agricultural or processed samples.

A system based on adsorption of gold nanoparticles on free antibody regions may function in an automated device configured to conduct sample preparation, reagent delivery, incubation, washing, and measurement without operator intervention. A system based on this technology may combine multiple quartz crystal supports to permit simultaneous detection of multiple analytes. A system based on this technology may operate within a distributed sensor network to monitor biological samples at remote locations.

Advantageously, the technology provides a detection mechanism that omits conjugation of gold nanoparticles with recognition molecules. This omission reduces consumption of antibodies, reduces preparation time for detection reagents, and avoids variability associated with conjugation chemistry. The technology provides a detection mechanism that relies on non-specific adsorption of gold nanoparticles on free antibody regions. This mechanism converts a source of background noise into a functional measurement parameter and thus reduces the impact of non-specific interactions on measurement accuracy.

The technology provides a measurement process that yields a negative correlation between analyte concentration and mass loading on a quartz crystal support. This correlation simplifies interpretation of mass-sensitive signals and permits use of straightforward calibration procedures. The technology provides a signal-enhancement method based on catalyzed deposition of silver on adsorbed gold nanoparticles. This method increases mass loading on the quartz crystal support and produces a frequency shift with improved resolution.

The technology provides a platform that accommodates a broad range of antibody-antigen pairs. This platform supports detection of proteins, peptides, nucleic acids, haptens, lipids, or other molecular targets when appropriate capture agents are immobilized on the quartz crystal support. The technology provides a sensing architecture that operates with microfluidic delivery of reagents. This architecture reduces reagent volume, reduces incubation time, and supports integration into automated devices.

The technology provides a measurement approach that operates without fluorescent labels, enzymatic labels, or radioactive labels. This approach reduces assay complexity and reduces dependence on optical instrumentation. The technology provides compatibility with quartz crystal microbalance instruments that deliver mass-sensitive signals with high temporal resolution. This compatibility permits real-time monitoring of reagent adsorption, analyte binding, and silver-enhancement progression.

The technology provides a reusable sensor surface. The immobilized antibody layer may be regenerated after completion of a detection cycle, enabling multiple uses of a single quartz crystal support under suitable regeneration conditions. The technology provides analytical results that exhibit improved reproducibility due to elimination of variability associated with antibody-nanoparticle conjugation. The technology provides a pathway for automated analysis through software control of fluid delivery, temperature regulation, signal acquisition, and data processing.

What is claimed is:

1. A kit for detecting or measuring the level of a target analyte in a biological sample, the kit comprising:
a solid support;
a capture antibody; and
gold nanoparticles (AuNPs).

2. The kit of claim 1, further comprising a solution comprising silver ions.

3. The kit of claim 1, further comprising a reducing reagent.

4. The kit of claim 1, wherein the AuNPs are 2 nm to 15 nm, 3 nm to 10 nm, or 4 nm to 7 nm in diameter.

5. The kit of claim 1, wherein the AuNPs are about 5 nm in diameter.

6. The kit of claim 1, wherein the AuNPs are tannic acid stabilized.

7. The kit of claim 1, wherein the solid support is a quartz crystal support.

8. The kit of claim 1, wherein the quartz crystal support is configured in a flow cell.

9. The kit of claim 1, wherein the quartz crystal support is suitable for use with a quartz crystal microbalance (QCM) instrument.

10. The kit of claim 1, further comprising a blocking agent.

11. The kit of claim 10, wherein the blocking agent comprises bovine serum albumin (BSA).

12. The kit of claim 1, wherein the capture antibody is of the IgG class.

13. A computer program product stored on non-transitory machine-readable media, the computer program product comprising machine-readable instructions for controlling a process for detecting or measuring the amount of an analyte in a sample, the process including control of flow of reagents to be applied to a solid support with the reagents including at least gold nanoparticles.

14. The computer program product of claim 13, wherein the process further includes control of flow of a solution comprising silver ions, optionally wherein the solution comprising silver ions further comprises a reducing reagent.

15. The computer program product of claim 13, wherein the analyte is an antibody, and wherein the process comprises instructions for measuring the level of the antibody.

16. The computer program product of claim 13, wherein the solid support is a quartz crystal microbalance (QCM) sensor and the instructions include a process for controlling operation of the QCM sensor.

17. The computer program product of claim 16, wherein the instructions include a process for measuring changes in the frequency of the QCM sensor based on a loading mass.

18. The computer program product of claim 13, wherein the instructions include process steps including applying a sample to be analyzed, applying a reagent including gold nanoparticles, and applying a reagent including silver ions, optionally wherein the reagent including silver ions further includes a reducing reagent.

19. The computer program product of claim 18, wherein the process steps further include operating a QCM sensor to measure a level of the analyte.

20. A system for detecting or measuring the level of a target analyte in a biological sample, the system comprising:
a solid support with a capture antibody attached thereto; and
a set of reagents including a solution containing gold nanoparticles and a solution containing silver ions;
wherein the capture antibody is selected to bind to a target antibody of interest.
